(19) United States
(12) Patent Application Publication
(10) Pub. No.: US 2019/0148006 A1
(43) Pub. Date: May 16, 2019

(54) REGISTRY AND IMAGE DATA ANALYTICS (RIDA) SYSTEM

(71) Applicant: MedStreaming, LLC, Redmond, WA (US)

(72) Inventors: Ahmed Aly Saad AHMED, Redmond, WA (US); Wael ELSEAIDY, Redmond, WA (US); Ewald de VRIES, Redmond, WA (US)

(21) Appl. No.: 16/192,782

(22) Filed: Nov. 15, 2018

Related U.S. Application Data
(60) Provisional application No. 62/586,882, filed on Nov. 15, 2017.

(51) Int. Cl. G16H 30/40; G06F 17/30; G16H 50/20; A61B 6/00; A61B 8/08; A61B 5/00

(52) U.S. Cl. CPC G16H 30/40; G06F 17/3028; A61B 5/7282; A61B 6/5205; A61B 8/5207; G16H 50/20

(57) ABSTRACT

Techniques are described for analyzing patient image data relative to stored medical image data. A Registry & Image Data Analytics (RIDA) system is described that is configured to analyze medical image data, generate corresponding metadata, and selectively store the medical image data, and metadata, within a registry & image data repository. The RIDA system may be configured to extract pertinent medical data from medical image data that is related to patient care, medical examinations and/or medical research. In doing so, the RIDA system may infer a medical diagnosis based on an analysis of patient image data. Additionally, the RIDA system may infer the validity of a medical hypothesis based at least in part on an analysis of patient image data. The RIDA system may also generate and store a synthesized expression of a text-based or audio-based user input into patient image data and/or corresponding registry data record.

### RELATED APPLICATIONS

This application claims priority to a co-pending, commonly owned U.S. Provisional Patent Application No. 62/586,882 filed on Nov. 15, 2017, and titled “Registry and Image Data Analytics (RIDA) System,” which is herein incorporated by reference in its entirety.

### BACKGROUND

Present day, clinical data management is becoming an increasingly crucial aspect of facilitating patient-specific care and conducting research to understand different abnormality and disease processes across populations. Medical practitioners are often burdened with the task of navigating through onerous and complicated information infrastructure. Often, a complete patient history may be difficult to discern because of the plethora of medical data, and format in which it is presented. While collecting raw data from disparate systems is only the beginning, present-day systems often lack technical capabilities in distilling the information in such a way that it may focus on specific questions or strategies that enable a medical practitioner to discover more meaningful insights that relate to patient-specific care, or population trends when dealing with different abnormalities and diseases.

### BRIEF DESCRIPTION OF THE DRAWINGS

The detailed description is set forth with reference to the accompanying figures. In the figures, the left-most digit(s) of a reference number identifies the figure in which the reference number first appears. The use of the same reference numbers in different figures indicates similar or identical items or features.

FIG. 1 illustrates a schematic view of a computing environment that facilitates an analysis of registry and patient image data via a Registry & Image Data Analytics (RIDA) system.

FIG. 2 illustrates a block diagram of a RIDA system process for analyzing a user input data via a client device, and further automating an inclusion of a text-based feature and/or graphics-based feature within patent image data and/or a corresponding registry data record.

FIG. 3 illustrates a block diagram of various components of the RIDA system.

FIG. 4 illustrates a block diagram of various components of a client device configured to interact with the RIDA system.

FIG. 5 illustrates a RIDA system process for inferring a medical diagnosis based on patent image data.

FIG. 6 illustrates a RIDA system process for validating a medical hypothesis associated with patient image data.

FIG. 7 illustrates a RIDA system process for analyzing medical image data and selectively storing the medical image data, along with corresponding metadata, in a data repository.

FIG. 8 illustrates a RIDA system process for synthesizing a user input to modify patient image data and/or a corresponding registry data record.

### DETAILED DESCRIPTION

This disclosure describes techniques for analyzing patient image data relative to a repository of stored medical image data. More specifically, a Registry & Image Data Analytics (RIDA) system is described that is configured to analyze medical image data, generate corresponding metadata, and selectively store the medical image data, along with corresponding metadata, within the registry & image data repository. Moreover, the RIDA system may be further configured to infer a diagnostic interpretation of a medical condition based on an automated analysis of patient medical data relative to a set of stored medical image data.

In various examples, the patient image data may be part of a medical examination and/or medical research, and include medical result data from an x-ray scan, magnetic resonance imaging (MRI) scan, computed tomography (CT) scan, ultrasound scan, or a Positron Emission Tomography (PET) scan. Further, the patient image data may include a graphical depiction of anatomical features of an organ system or graphics-based result data. The stored medical image data may correspond to patient image data. However, rather than being attributed to a particular patient, stored medical image data may be associated with a specified control group of patients of varying size.

This disclosure describes techniques whereby the RIDA system may be configured to extract pertinent medical data from the medical image data. Pertinent medical data may comprise text data and image data that is related to the medical examination and/or medical research. Further, the pertinent medical data may include a patient identifier, a medical imaging technique identifier, an organ system identifier, an anatomical feature identifier, a date and time of when the medical image data was captured, or any combination thereof. In some examples, the RIDA system may employ object recognition algorithms, such as appearance-based and feature-based methods—to help identify pertinent medical characteristics from the text and images of the medical image data. The set of metadata may include a patient identifier, organ system identifier, medical specialty identifier, medical imaging technique identifier, examination identifier, and patient demographic identifiers, or any combination thereof. In doing so, the RIDA system may associate the set of metadata with each corresponding instance of medical image data. The term “medical image data” may refer interchangeably to patient image data and stored medical image data. In some examples, the RIDA system may selectively remove patient identifiers presented in the medical image data to safeguard patient privacy and ensure that any distribution of the medical image data, for research or other related purposes, does not inadvertently reveal a patient's identity.

Moreover, the RIDA system may be configured to analyze a user input to modify a text-based or a graphics-based feature associated with patient image data and/or a corresponding registry data record. The user input may correspond to text-based data, audio-based data, or a combination of both. More specifically, the RIDA system may analyze the user input using natural language processing (NLP) and natural language understanding (NLU) algorithms, or a data mining algorithm, to determine a context and command of the user input. The context and command may relate to whether the modification or annotation is associated with a text-based feature or a graphics-based feature of the patient image data and/or a corresponding registry data record. In doing so, the RIDA system may generate and store a synthesized expression of the user input in the patient image data and/or corresponding registry data record.

Further, the RIDA system may be configured to infer a medical diagnosis based on patient image data and infer whether a medical hypothesis is valid based at least in part on patient image data. Specifically, the RIDA system may employ a data mining algorithm to extract pertinent medical data from the patient image data. The data mining algorithm may extract words, terms, phrases, quotes from the input medical image data.

Moreover, the RIDA system may identify and retrieve a set of stored medical image data based on a similarity with the set of pertinent medical data. Further, the RIDA system may employ one or more trained machined learning algorithms to analyze a set of image features of the patient image data to identify data patterns with the set of stored medical image data and further infer a medical diagnosis associated with the patient image data.

In another example, the RIDA system may receive a request to validate a medical hypothesis associated with patient image data. In some examples, the request may include a set of control group criteria and a set of analysis criteria. The set of control group criteria may identify profile data that may be used to establish a control group of patients that are associated with the medical hypothesis. The set of analysis criteria may include one or more test algorithms that are configured to test the medical hypothesis. Alternatively, the set of analysis criteria may comprise filter criteria that may be used to compile a subset of pertinent medical data. For example, the set of analysis criteria may filter the set of medical image data to include only those related to a particular condition.

The RIDA system may retrieve a set of stored medical image data that is based on the set of control group criteria, and further, analyze image features of the set of stored medical image data based at least in part on the set of analysis criteria. In one example, the RIDA system may employ one or more machine-learning algorithms to analyze the set of stored medical image data using the one or more test algorithms associated with the set of analysis criteria. In doing so, the RIDA system may assign each individual analysis of a test algorithm with a validity score. The validity score may represent whether or not a test algorithm validated a medical hypothesis using the set of stored medical image data (i.e. a set of control group criteria).

Additionally, the RIDA system may generate one or more hypothesis rules based on the medical hypothesis. Each hypothesis rule may correlate the validity of the medical hypothesis with a subset of pertinent medical data. In this example, the RIDA system may analyze the set of image features of the set of stored medical image data based at least in part on the one or more hypothesis rules. The RIDA system may associate a validity score to each analysis of a hypothesis rule and a set of stored medical image data for the purpose of determining whether the medical hypothesis is validated by each of hypothesis rule.

In another example, the RIDA system may employ one or more machine-learning algorithms to generate a statistical data model that aggregates a set of stored medical image data associated with a predetermined patient population captured over a predetermined time interval. The predetermined patient population may include the control group established by the set of control group criteria and may be equal to or greater than the control group, in size. In doing so, the RIDA system may infer the validity of the medical hypothesis using the statistical data model and further based at least in part on the set of control group criteria and the set of analysis criteria. In this example, the set of control group criteria corresponds to a control group that is a subset of the predetermined patient population used to generate the statistical data model. A benefit of using a statistical model is an ability to modify a set of control group criteria in the event that an initial set of control group criteria did not validate the medical hypothesis. For example, the RIDA system may use one or more machine-learning algorithms to modify the set of control group criteria, based on data patterns within the statistical model. The RIDA system may then retrieve an additional set of stored medical image data associated with the modified set of control group criteria, and then inferring a validity of the medical hypothesis based at least in part on an additional analysis of the additional set of stored medical image data.

Further, the term “techniques,” as used herein, may refer to system(s), method(s), computer-readable instruction(s), module(s), algorithms, hardware logic, and/or operation(s) as permitted by the context described above and through the document.

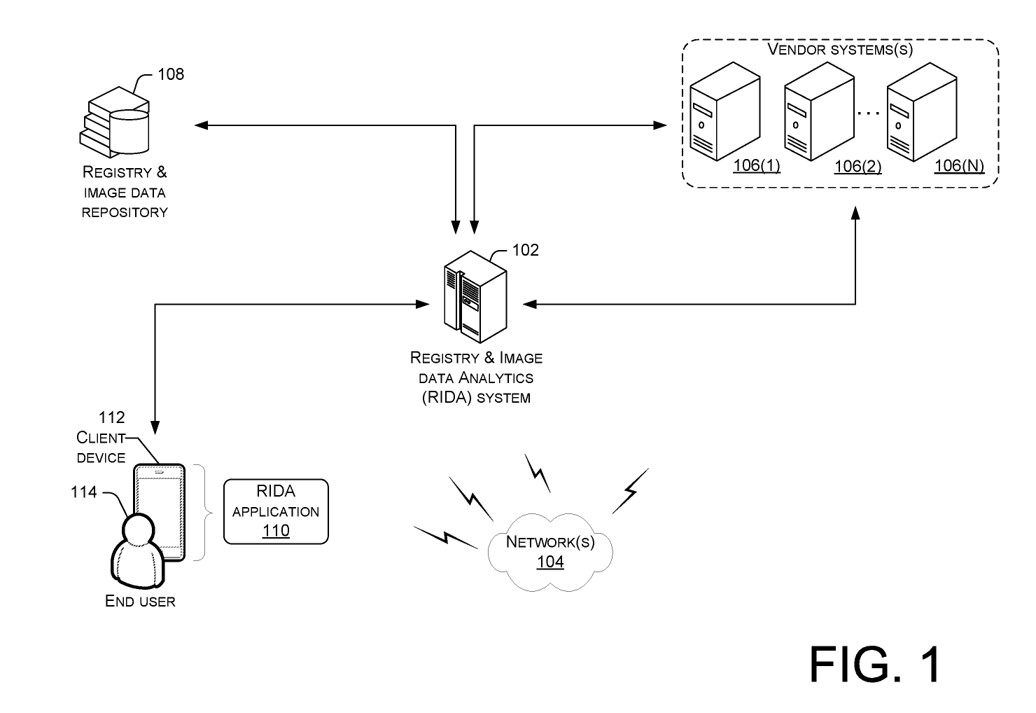

FIG. 1 illustrates a schematic view of a computing environment 100 that facilitates an analysis of registry and patient image data via a Registry & Image Data Analytics (RIDA) system 102. In various examples, the RIDA system 102 is configured to communicate, via one or more network(s) 104, with one or more vendor system(s) 106(1)-106(N), a registry & image data repository 108, a RIDA application 110 native on a client device 112, or any combination thereof.

In the illustrated example, the RIDA system 102 may operate on one or more distributed computing resource(s). The distributed computing resource(s) may include one or more computing device(s) that operate in a cluster or other configuration to share resources, balance load, increase performance, provide fail-over support or redundancy, or for other purposes.

The one or more vendor system(s) 106(1)-106(N) may include any medical or research organization or individual for that matter, that may be interested in analyzing medical image data. Further, the client device 112 may be associated with an end user 114 that is associated with the one or more vendor system(s) 106(1)-106(N).

The RIDA system 102 may be configured to analyze medical image data, generate corresponding metadata, and selectively store the medical image data, along with corresponding metadata, within the registry & image data repository 108. The medical image data may relate to image data associated with patient care, a medical examination, and/or medical research. In various example, the medical image data may include an x-ray scan, a magnetic resonance imaging (MRI) scan, a computed tomography (CT) scan, an ultrasound scan, or a Positron Emission Tomography (PET) scan.

Further, the corresponding metadata may include patient identifier, organ system identifier, medical specialty identifier, medical imaging technique identifier, examination identifier, and patient demographic identifiers, or any combination thereof.

The registry & image data repository 108 may include a patient data-store and an anonymized data-store. The anonymized data-store may include substantially similar registry data as the patient data-store, but for patient identifiers. The purpose for doing so is to safeguard patient privacy when registry and image data is used for research or other related purposes.

Moreover, the RIDA system 102 may be further configured to infer a diagnostic interpretation of a medical condition based on an automated analysis of patient medical data relative to a set of stored medical image data. The RIDA system 102 may retrieve the set of stored medical image data from a registry and image data repository 108 that is accessible by the RIDA system. Additionally, the RIDA system 102 may infer the validity of a medical hypothesis for a medical condition, via an analysis of patient image data relative to a set of stored medical image data. In this example, medical researchers may use the RIDA system 102, and registry data within the registry & image data repository 108, as a platform to upload proprietary data and test algorithms associated with medical hypotheses.

Additionally, the RIDA system 102 may analyze a user input that is received via a RIDA application 110 native on a client device 112 and further automate and store a synthesized expression on a corresponding instance of patient image data. The user input may be text-based, audio-based, or a combination of both. Further, the synthesized expression may correspond to an inclusion or manipulation of a text-based feature within patient image data and/or a corresponding registry data record. Text-based features may include annotations associated with a diagnostic interpretation of medical image data and/or medical results. Alternatively, or additionally, the synthesized expression may correspond to an inclusion or manipulation of a graphics-based feature within patent image data and/or a corresponding registry data record. Graphics-based features may include schematic illustrations of an organ system, such as a patient's cardiovascular system, and/or medical results. such as a schematic diagram of a patient's organ system.

In the illustrated example, the one or more network(s) 104 may include public networks such as the Internet, private networks such as an institutional and/or personal intranet, or some combination of private and public networks. The one or more network(s) 104 can also include any type of wired and/or wireless network, including but not limited to local area network (LANs), wide area networks (WANs), satellite networks, cable networks, Wi-Fi networks, Wi-Max networks, mobile communications networks (e.g. 3G, 4G, and so forth), or any combination thereof.

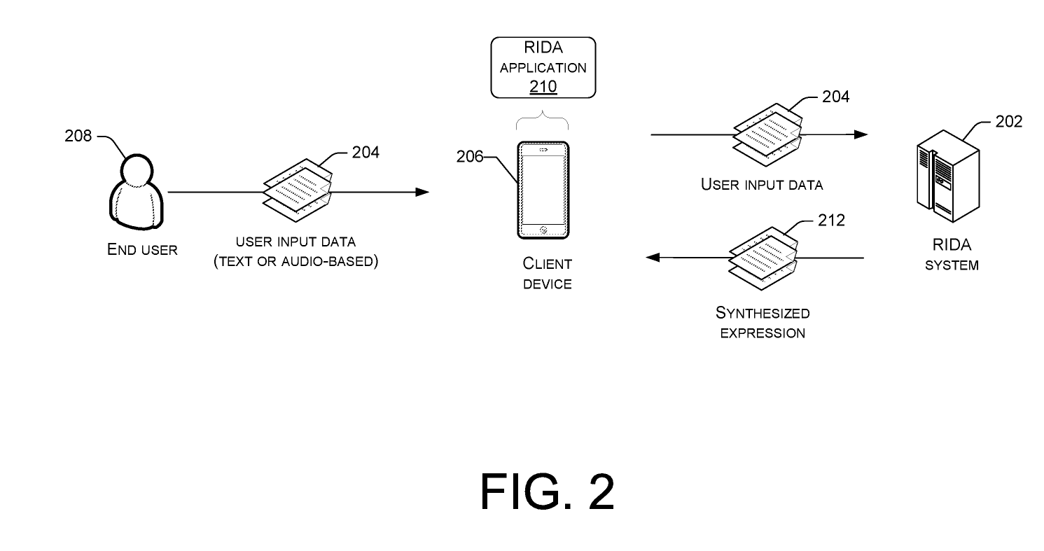

FIG. 2 illustrates a block diagram of a RIDA system 202 process for analyzing a user input data 204 via a client device 206, and further automating an inclusion of a text-based feature and/or graphics-based feature within patent image data and/or a corresponding registry data record. In the illustrated example, the end user 208 may transmit user input data 204 to the RIDA system 202. The end user 208 may transmit the user input data 204 via a RIDA application 210 that is native on the client device 206. In some examples, the user input data 204 may correspond to text-based data, audio-based data, or a combination of both.

In some examples, the RIDA system 202 may employ a data mining algorithm to extract words, terms, phrases, and quotes from a text-based user input. The data mining algorithm may use both machine learning and non-machine learning techniques to determine a corresponding context and command. In doing so, the RIDA system 202 may automatically generate a synthesized expression 212, based on the text-based user input. In doing so, the synthesized expression 212 may be incorporated within an updated iteration of the patient image data, and/or a corresponding registry data record, and further presented to the end user 208 via the RIDA application 210 native on the client device 206. In one example, the synthesized expression 212 may correspond to an inclusion of a text-based feature within the patient image data or corresponding registry data record. The text-based feature may correspond to a diagnostic interpretation of medical image data. Alternatively, or additionally, the synthesized expression 212 may correspond to manipulating a graphics-based feature associated with the registry data record. The graphics-based feature may correspond to a schematic diagram of an organ system, and the synthesized expression 212 may correspond to graphically representing a medical condition on the schematic diagram. In the latter example, consider patient image data and/or a corresponding registry data record that includes a schematic illustration of a patient's cardiovascular system. In this example, the synthesized expression 212 may reflect a predetermined percentage occlusion of a carotid artery at a specific location on the schematic illustration.

Additionally, the RIDA system may employ natural language processing (NLP) and natural language understanding (NLU) algorithms to parse through an audio-based user input. In doing so, the RIDA system 202 may automatically generate a synthesized expression 212, based on the audio-based user input. As discussed earlier, the synthesized expression 212 may correspond to an inclusion of a text-based feature or a graphics-based feature within the registry data record.

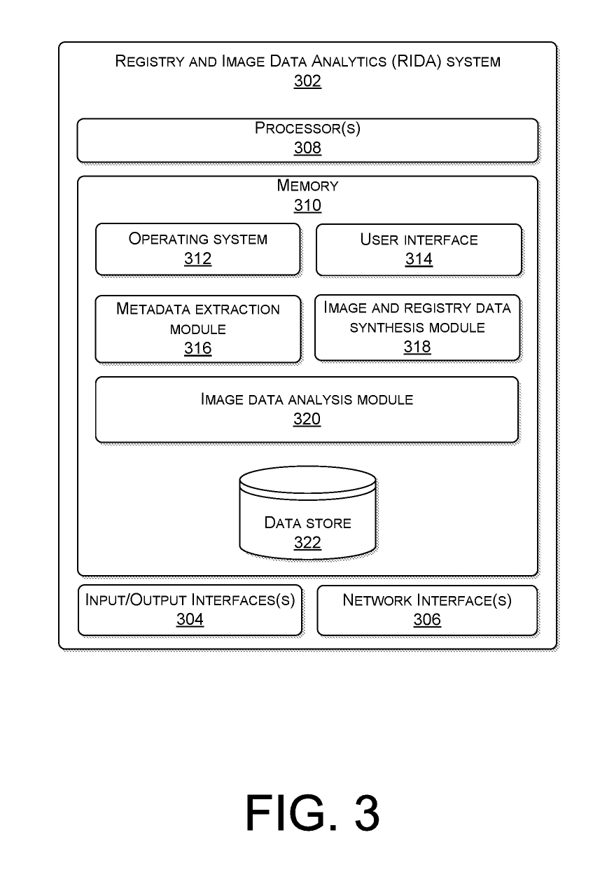

FIG. 3 illustrates a block diagram of various components of the RIDA system 302. In various examples, the RIDA system 302 is configured to analyze medical image data, generate corresponding metadata, and selectively store the medical image data, along with corresponding metadata, within the registry & image data repository 108. Moreover, the RIDA system 302 may be further configured to infer a diagnostic interpretation of a medical condition based on an automated analysis of patient medical data relative to a set of stored medical image data.

The RIDA system 302 may include input/output interface(s) 304. The input/output interface(s) 304 may include any type of output interface known in the art, such as a display (e.g. a liquid crystal display), speakers, a vibrating mechanism, or a tactile feedback mechanism. Input/output interface(s) 304 also include ports for one or more peripheral devices, such as headphones, peripheral speakers, or a peripheral display. Further, the input/output interface(s) 304 may further include a camera, a microphone, a keyboard/keypad, or a touch-sensitive display. A keyboard/keypad may be a push button numerical dialing pad (such as on a typical telecommunication device), a multi-key keyboard (such as a conventional QWERTY keyboard), or one or more other types of keys or buttons, and may also include a joystick-like controller and/or designated navigation buttons, or the like.

Additionally, the RIDA system 302 may include network interface(s) 306. The network interface(s) 306 may include any sort of transceiver known in the art. For example, the network interface(s) 306 may include a radio transceiver that performs the function of transmitting and receiving radio frequency communications via an antenna. In addition, the network interface(s) 306 may also include a wireless communication transceiver and a near-field antenna for communicating over unlicensed wireless Internet Protocol (IP) networks, such as local wireless data networks and personal area networks (e.g. Bluetooth or near field communication (NFC) networks). Further, the network interface(s) 306 may include wired communication components, such as an Ethernet port or a Universal Serial Bus (USB).

Further, the RIDA system 302 may include one or more processor(s) 308 that are operably connected to memory 310. In at least one example, the one or more processor(s) 308 may be a central processing unit(s) (CPU), graphics processing unit(s) (GPU), or both a CPU and GPU or any other sort of processing unit(s). Each of the one or more processor(s) 308 may have numerous arithmetic logic units (ALUs) that perform arithmetic and logical operations as well as one or more control units (CUs) that extract instructions and stored content from processor cache memory, and then executes these instructions by calling on the ALUs, as necessary during program execution. The one or more processor(s) 308 may also be responsible for executing all computer applications stored in the memory, which can be associated with common types of volatile (RAM) and/or non-volatile (ROM) memory.

In some examples, memory 310 may include system memory, which may be volatile (such as RAM), non-volatile (such as ROM, flash memory, etc.) or some combination of the two. The memory may also include additional data storage devices (removable and/or non-removable) such as, for example, magnetic disks, optical disks, or tape.

The memory 310 may further include non-transitory computer-readable media, such as volatile and nonvolatile, removable and non-removable media implemented in any method or technology for storage of information, such as computer readable instructions, data structures, program modules, or other data. System memory, removable storage, and non-removable storage are all examples of non-transitory computer-readable media. Examples of non-transitory computer-readable media include, but are not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other non-transitory medium which can be used to store the desired information.

In the illustrated example, the memory 310 may include an operating system 312, a user interface 314, a metadata extraction module 316, an image and registry data synthesis module 318, an image data analysis module 320, and a data store 322. The operating system 312 may be any operating system capable of managing computer hardware and software resources. The operating system 312 may include an interface layer that enables applications to interface with the input/output interface(s) 304 and the network interface(s) 306. The interface layer may comprise public APIs, private APIs, or a combination of both. Additionally, the operating system 312 may include other components that perform various other functions generally associated with an operating system.

The user interface 314 may be configured to enable a user to provide inputs and receive outputs from the RIDA system 302. Example data inputs for a user may include user input data to modify or annotate patient image data and/or a corresponding registry data record. Other examples of data inputs may relate to inferring the validity of a medical hypothesis and include defining predetermined validity thresholds, sets of control group criteria, sets of analysis criteria, and a predetermined patient population for a statistical data model.

The metadata extraction module 316 may be configured to extract pertinent medical data from medical image data. The term “medical image data” may refer interchangeably to patient image data and stored medical image data. Pertinent medical data may comprise text data and image data that is related to the medical examination and/or medical research. Further, the pertinent medical data may include a patient identifier, a medical imaging technique identifier, an organ system identifier, an anatomical feature identifier, a date and time of when the medical image data was captured, or any combination thereof.

In some examples, metadata extraction module 316 may employ object recognition algorithms, such as appearance-based and feature-based methods—to help identify pertinent medical characteristics from the text and images of the medical image data.

Moreover, the metadata extraction module 316 may generate a set of metadata for the medical image data, based at least in part on an analysis of the pertinent medical data. The set of metadata may include a patient identifier, organ system identifier, medical specialty identifier, medical imaging technique identifier, examination identifier, and patient demographic identifiers, or any combination thereof. In doing so, the RIDA system may associate the set of metadata with each corresponding instance of medical image data.

The metadata extraction module 316 may selectively remove patient identifiers presented in the medical image data to safeguard patient privacy and ensure that any distribution of the medical image data, for research or other related purposes, does not inadvertently reveal a patient's identity.

The image and registry data synthesis module 318 may be configured to analyze a user input to modify a text-based or a graphics-based feature associated with patient image data and/or a corresponding registry data record. The user input may correspond to text-based data, audio-based data, or a combination of both. More specifically, the image and registry data synthesis module 318 may analyze the user input using natural language processing (NLP) and natural language understanding (NLU) algorithms, or a data mining algorithm, to determine a context and command of the user input. The context and command may relate to whether the modification or annotation is associated with a text-based feature or a graphics-based feature of the patient image data and/or a corresponding registry data record. In doing so, the image and registry data synthesis module 318 may generate and store a synthesized expression of the user input in the patient image data and/or corresponding registry data record.

The image data analysis module 320 may be configured to infer a medical diagnosis based on patient image data and infer whether a medical hypothesis is valid based at least in part on patient image data. The operations of the image data analysis module 320 are described in further detail with reference to processes 500 and 600. Specifically, the image data analysis module 320 may retrieve, from the data store 322, patient image data associated with patient care. The patient image data may be part of a medical examination and/or medical research, and include medical result data from an x-ray scan, magnetic resonance imaging (MM) scan, computed tomography (CT) scan, ultrasound scan, or a Positron Emission Tomography (PET) scan. Further, the patient image data may include a graphical depiction of anatomical features of an organ system or graphics-based result data.

The image data analysis module 320 may employ a data mining algorithm to extract pertinent medical data from the patient image data. The data mining algorithm may extract words, terms, phrases, quotes from the input medical image data. The data mining algorithm may use both machine learning and non-machine learning techniques such as decision tree learning, association rule learning, artificial neural networks, inductive logic, Support Vector Machines (SVMs), clustering, Bayesian networks, reinforcement learning, representation learning, similarity and metric learning, and sparse dictionary learning to extract the patterns.

Moreover, the image data analysis module 320 may identify and retrieve a set of stored medical image data from the data store 322 based on a similarity with the set of pertinent medical data. The similarity may be based on a medical imaging technique identifier, an organ system identifier, a patient identifier, or any combination thereof. The image data analysis module 320 may employ one or more trained machined learning algorithms to analyze a set of image features of the patient image data to identify data patterns with the set of stored medical image data. In this way, the image data analysis module 320 may infer a medical diagnosis associated with the patient image data, based at least in part on an analysis of the set of image features.

In another example, the image data analysis module 320 may receive a request to validate a medical hypothesis associated with patient image data. In some examples, the request may include a set of control group criteria and a set of analysis criteria. The set of control group criteria may identify profile data that may be used to establish a control group of patients that are associated with the medical hypothesis. The set of analysis criteria may include one or more test algorithms that are configured to test the medical hypothesis. Alternatively, the set of analysis criteria may comprise filter criteria that may be used to compile a subset of pertinent medical data. For example, the set of analysis criteria may filter the set of medical image data to include only those related to a particular condition.

The image data analysis module 320 may retrieve, from the data store 322, a set of stored medical image data that is based on the set of control group criteria, and further, analyze image features of the set of stored medical image data based at least in part on the set of analysis criteria. In one example, the image data analysis module 320 may employ one or more machine-learning algorithms to analyze the set of stored medical image data using the one or more test algorithms associated with the set of analysis criteria. In doing so, the image data analysis module 320 may assign each individual analysis of a test algorithm with a validity score. The validity score may represent whether or not a test algorithm validated a medical hypothesis using the set of stored medical image data (i.e. a set of control group criteria). The validity score may be alpha-numeric (i.e. 0 to 10, or A to F), descriptive (i.e. low, medium, or high), based on color, (i.e. red, yellow, or green), or any other suitable rating scale. In one example, a high validity score (i.e. 7 to 10, high, or green) may indicate a high likelihood that the test algorithm validates the medical hypothesis. In contrast, a low validity score (i.e. 0 to 3, low, or red) may indicate that the test algorithm is unlikely to validate the medical hypothesis.

Additionally, the image data analysis module 320 may generate one or more hypothesis rules based on the medical hypothesis. Each hypothesis rule may correlate the validity of the medical hypothesis with a subset of pertinent medical data. In this example, the image data analysis module 320 may analyze the set of image features of the set of stored medical image data based at least in part on the one or more hypothesis rules. Further, the image data analysis module 320 may associate a validity score with each analysis of a hypothesis rule and a set of stored medical image data for the purpose of determining whether the medical hypothesis is validated by each hypothesis rule.

In another example, the image data analysis module 320 may employ one or more machine-learning algorithms to generate a statistical data model that aggregates a set of stored medical image data associated with a predetermined patient population captured over a predetermined time interval. The predetermined patient population may include the control group established by the set of control group criteria and may be equal to or greater than the control group, in size. In doing so, the image data analysis module 320 may infer the validity of the medical hypothesis using the statistical data model and further based at least in part on the set of control group criteria and the set of analysis criteria. In this example, the set of control group criteria corresponds to a control group that is a subset of the predetermined patient population used to generate the statistical data model. A benefit of using a statistical model is an ability to modify a set of control group criteria in the event that an initial set of control group criteria did not validate the medical hypothesis. For example, the image data analysis module 320 may use one or more machine-learning algorithms to modify the set of control group criteria, based on data patterns within the statistical model. The image data analysis module 320 may then retrieve an additional set of stored medical image data associated with the modified set of control group criteria, and then inferring a validity of the medical hypothesis based at least in part on an additional analysis of the additional set of stored medical image data.

The data store 322 may include a patient data-store and an anonymized data-store. The anonymized data-store may include substantially similar registry data as the patient data-store, but for patient identifiers. The purpose for doing so is to safeguard patient privacy when registry and image data is used for research or other related purposes. The patient data-store and the anonymized data store may include stored medical image data for a predetermined population over a predetermined time interval. It is noteworthy that the patient image data may correspond to the stored medical image data.

The data store 322 may further include patient image data and/or corresponding registry data records. The patient image data may be part of a medical examination and/or medical research, and include medical result data from an x-ray scan, magnetic resonance imaging (MM) scan, computed tomography (CT) scan, ultrasound scan, or a Positron Emission Tomography (PET) scan. The registry data records may include medical annotations and/or medical interpretations that cross-reference and/or relate to the patient image data and/or a set of stored medical image data.

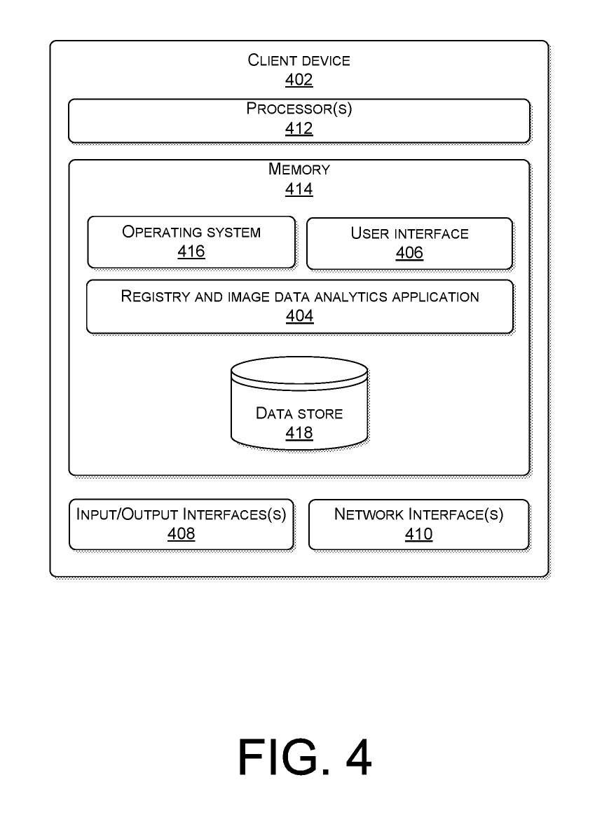

FIG. 4 illustrates a block diagram of various components of a client device configured to interact with the RIDA system 302. In various examples, client device 402, via a RIDA application 404, may be configured to transmit and receive patient image data and corresponding analyses via the RIDA system. In one example, the client device 402, via the RIDA application 404, may transmit user input data to the RIDA system 302 to facilitate modifying or annotating patient medical data, or corresponding registry data record. Further, the client device 402, via the RIDA application 404, may present, via a user interface 406, a synthesized expression of the patient medical data, or corresponding registry data record that is modified in accordance with the user input data.

In the illustrated example, the client device 402 may include input/output interface(s) 408 and network interface(s) 410. The input/output interface(s) 408 may be similar to the input/output interface(s) 304, and the network interface(s) 410 may be similar to the network interface(s) 306.

Further, the client device 402 may include one or more processor(s) 412 that are operably connected to memory 414. The one or more processor(s) 412 may be similar to the one or more processor(s) 308 and the memory 414 may be similar to the memory 310.

In the illustrated example, the memory 414 may include an operating system 416, the user interface 406, the RIDA application 404, and a data store 424. The operating system 416 may be similar to the operating system 312.

The user interface 406 may enable a user to provide inputs and receive outputs from the RIDA system 302. Example data inputs for a user may include user input data to modify or annotate patient image data and/or a corresponding registry data record. Other examples of data inputs may relate to inferring the validity of a medical hypothesis and include defining predetermined validity thresholds, sets of control group criteria, sets of analysis criteria, and a predetermined patient population for a statistical data model.

In various examples, the RIDA application 404 may be configured to present patient image data and corresponding analyses on the user interface 406 of the client device 402. In one example, the RIDA application 404 may enable an end user to transmit user input data to the RIDA system 302 to modify or annotate patient medical data, or corresponding registry data record. Further, the RIDA application 404, may present, via the user interface 406, a synthesized expression of patient medical data, or corresponding registry data record that is modified in accordance with the user input data. Moreover, the RIDA application 404 may facilitate a user input of various parameters relating to inferring the validity of a medical hypothesis, such as predetermined validity thresholds, sets of control group criteria, sets of analysis criteria, and a predetermined patient population for a statistical data model.

Moreover, the data store 418 may correspond to the registry and image data repository 108 and include a patient data-store and/or an anonymized data-store. The anonymized data-store may include substantially similar registry data as the patient data-store, but for patient identifiers. The purpose for doing so is to safeguard patient privacy when registry and image data is used for research or other related purposes.

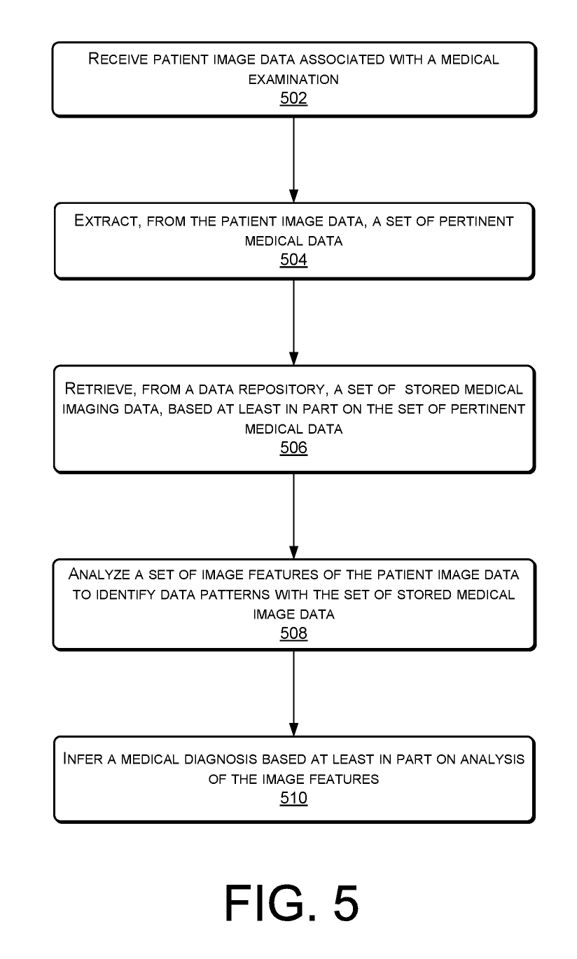

FIGS. 5 through 9 and present processes 500 through 900 that relate to operations of the Registry and Image Data Analytics (RIDA) system. Each of processes 500 through 900 illustrate a collection of blocks in a logical flow chart, which represents a sequence of operations that can be implemented in hardware, software, or a combination thereof. In the context of software, the blocks represent computer-executable instructions that, when executed by one or more processors, perform the recited operations. Generally, computer-executable instructions may include routines, programs, objects, components, data structures, and the like that perform particular functions or implement particular abstract data types. The order in which the operations are described is not intended to be construed as a limitation, and any number of the described blocks can be combined in any order and/or in parallel to implement the process. For discussion purposes, the processes 500 through 900 are described with reference to the computing environment 100 of FIG. 1.

FIG. 5 illustrates a RIDA system process for inferring a medical diagnosis based on patient image data. More specifically the RIDA system may use one or more trained machine learning algorithms to correlate patient image data with stored medical image data.

At 502, the RIDA system may receive patient image data associated with patient care. The patient image data may be part of a medical examination and/or medical research, and include medical result data from an x-ray scan, magnetic resonance imaging (MRI) scan, computed tomography (CT) scan, ultrasound scan, or a Positron Emission Tomography (PET) scan. Further, the patient image data may include a graphical depiction of anatomical features of an organ system or graphics-based result data.

At 504, the RIDA system may extract pertinent medical data from the patient image data. The pertinent medical data may include a patient identifier, a medical imaging technique identifier, an organ system identifier, an anatomical feature identifier, a date and time of when the medical image data was captured, or any combination thereof.

In one example, the RIDA system may employ a data mining algorithm to extract pertinent medical data from the patient image data. The data mining algorithm may extract words, terms, phrases, quotes from the input medical image data. The data mining algorithm may use both machine learning and non-machine learning techniques such as decision tree learning, association rule learning, artificial neural networks, inductive logic, Support Vector Machines (SVMs), clustering, Bayesian networks, reinforcement learning, representation learning, similarity and metric learning, and sparse dictionary learning to extract the patterns.

At 506, the RIDA system may retrieve a set of stored medical image data from a data repository accessible by the RIDA system, based at least in part on a similarity to the set of pertinent medical data. For example, the RIDA system may identify the set of stored medical image data based on a similarity with the set of pertinent medical data. The similarity may be based on a medical imaging technique identifier, an organ system identifier, a patient identifier, or any combination thereof. In some examples, a user of the RIDA system may prescribe criteria by which the set of stored medical image data is selected. Criteria may include factors such as a control group of patients, a demographic profile, or any other medical or non-medical data that may be common among instances of the set of stored medical image data.

At 508, the RIDA system may analyze a set of image features of the patient image data to identify data patterns with the set of stored medical image data. The set of image features may include a visual depiction of anatomical features of an organ system along with overlaid pertinent medical characteristics. For example, with regards to an ultrasound test of a vascular system, pertinent medical characteristics may include stenosis, blood flow velocity (i.e. PSV and/or ESV), and/or plaque characteristics of a carotid artery. In some examples, the RIDA system may employ one or more trained machined learning algorithms to identify data patterns between the patient image data and the set of stored medical image data.

At 510, the RIDA system may infer a medical diagnosis associated with the patient image data, based at least in part on an analysis of the set of image features. In one example, the RIDA system may infer the medical diagnosis based on analysis of text data within the patient image data and/or the set of stored medical image data. Alternatively, or additionally, the RIDA system may access registry data records associated with the set of stored medical image data, and further base an inference of the medical diagnosis on the registry data records. The registry data records may be stored with a data repository accessible by the RIDA system. Further, the registry data records may include medical annotations and/or medical interpretations that cross-reference and/or relate to the set of stored medical image data.

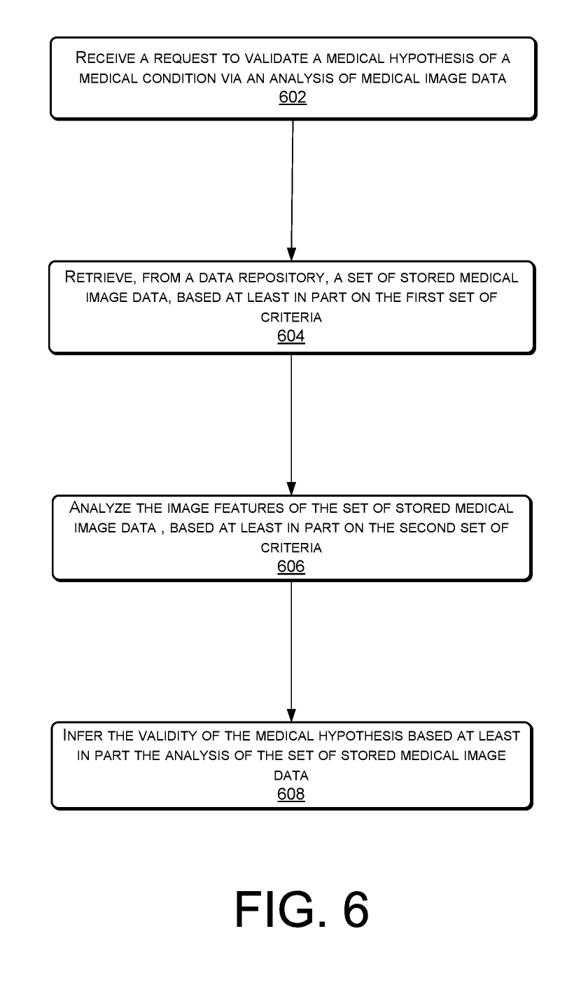

FIG. 6 illustrates a RIDA system process for validating a medical hypothesis associated with patient image data. A medical hypothesis, as used herein, purports a relationship between a medical diagnosis and diagnostic data within a control group being studied. The diagnostic data may relate to medical results data or profile data associated with patients within the control group being studied. In any case, the term medical hypothesis is intended to surmise a commonality among patients within the control group that share a common medical diagnosis. In various examples, the RIDA system may validate or invalidate a medical hypothesis. In some examples, the RIDA system may employ one or more machine-learning algorithms to modify a prescribed control group that is used to validate or invalidate, a medical hypothesis.

At 602, the RIDA system may receive a request to validate a medical hypothesis associated with patient image data. In some examples, the request may include a set of control group criteria and a set of analysis criteria. The set of control group criteria may identify profile data that may be used to establish a control group of patients that are associated with the medical hypothesis. The set of analysis criteria may include one or more test algorithms that are configured to test the medical hypothesis. For example, a test algorithm may presuppose a relationship between pertinent medical data, and in doing so, the RIDA system may be used a platform to validate a medical hypothesis using a test algorithm on the control group of patients established by the set of control group criteria. In various examples, various vendors (i.e. medical facilities, medical research facilities, or any other clinical or biomedical research facility) may their own proprietary test algorithms to the RIDA system for the purpose of validating a medical hypothesis. The RIDA system may employ one or more machine-learning algorithms to simultaneously analyze each of the test algorithms to determine whether one or more successfully validate the medical hypothesis.

Alternatively, the set of analysis criteria may comprise filter criteria that may be used to compile a subset of pertinent medical data. For example, the set of analysis criteria may filter the set of medical image data to include only those related to a particular condition. By way of example, the particular condition may be a percentage of stenosis, or occlusion, due to plaque build up at a particular point along a carotid artery.

At 604, the RIDA system may retrieve, from a data repository accessible by the RIDA system, a set of stored medical image data that is based on the set of control group criteria. In other words, in this step, the RIDA system is establishing a control group to test the validity of the medical hypothesis. In one example, the set of control group criteria may relate to patient demographics, anatomical features of an organ system, pertinent medical characteristics, medical conditions, or any combination thereof.

At 606, the RIDA system may analyze the image feature of the set of stored medical image data based at least in part on the set of analysis criteria. In one example, the RIDA system may employ one or more machine-learning algorithms to analyze the set of stored medical image data using the one or more test algorithms associated with the set of analysis criteria. In doing so, the RIDA system may assign each individual analysis of a test algorithm with a validity score. The validity score may represent whether or not a test algorithm validated a medical hypothesis using the set of stored medical image data (i.e. a set of control group criteria).

In another non-limiting example, the RIDA system may generate one or more hypothesis rules based on the medical hypothesis. Each hypothesis rule may correlate the validity of the medical hypothesis with a subset of pertinent medical data. In this example, the RIDA system may analyze the set of image features of the set of stored medical image data based at least in part on the one or more hypothesis rules. Further, the RIDA system may associate a validity score with each analysis of a hypothesis rule and a set of stored medical image data for the purpose of determining whether the medical hypothesis is validated.

At 608, the RIDA system may infer the validity of the medical hypothesis based at least in part on the analysis of the set of stored medical image data. In one example, the RIDA system may infer that one or more test algorithms validate a medical hypothesis based at least in part on corresponding validity scores being greater than or equal to the predetermined validity threshold. The RIDA system may employ one or more machine-learning algorithms to execute a plurality of test algorithms simultaneously. In doing so, the RIDA system may identify a subset of individual analyses that have validity scores greater than or equal to a predetermined validity threshold. The predetermined validity threshold may be set by an operator of the RIDA system or any other administrator with an interest in the validity of the medical hypothesis.

In another example, the RIDA system may employ one or more machine-learning algorithms to generate a statistical data model that aggregates a set of stored medical image data associated with a predetermined patient population captured over a predetermined time interval. The predetermined patient population may include the control group established by the set of control group criteria and may be equal to or greater than the control group, in size. In doing so, the RIDA system may infer the validity of the medical hypothesis using the statistical data model and further based at least in part on the set of control group criteria and the set of analysis criteria. In this example, the set of control group criteria corresponds to a control group that is a subset of the predetermined patient population used to generate the statistical data model.

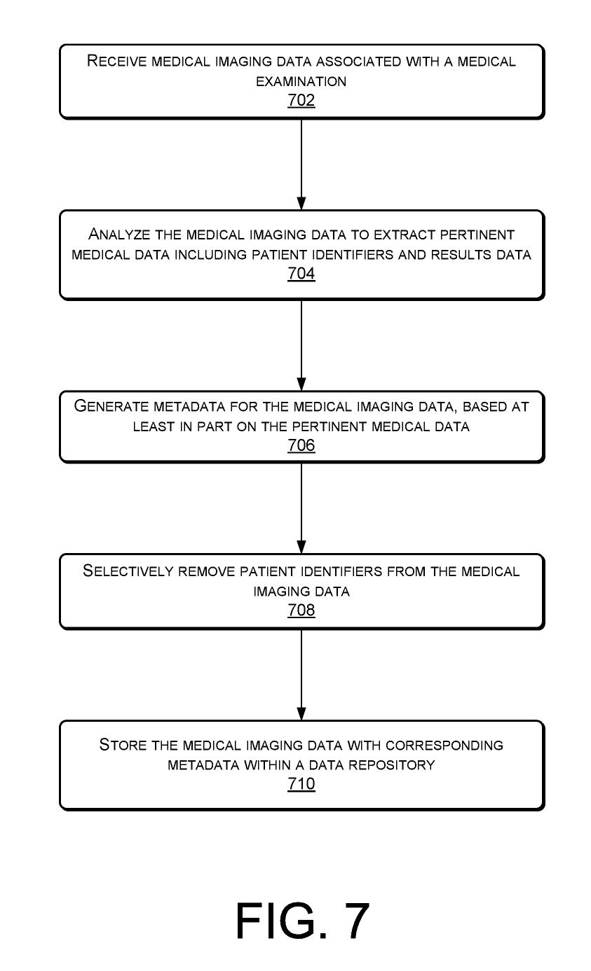

FIG. 7 illustrates a RIDA system process for analyzing medical image data and selectively storing the medical image data, along with corresponding metadata, in a data repository. In one example, the data repository may be a patient identifiable information. Alternatively, the data repository may be an anonymized repository, whereby patient identifiable information is removed.

At 702, the RIDA system may receive medical image data associated with a medical examination and/or medical research. The medical image data may include image data associated with patient care. The image data may be further related to one of an x-ray scan, magnetic resonance imaging (Mill) scan, computed tomography (CT) scan, ultrasound scan, or a Positron Emission Tomography (PET) scan. Further, the RIDA system may receive the medical image data from one or more vendor(s), including medical facilities, medical research facilities, or any other clinical or biomedical research facility.

At 704, the RIDA system may extract pertinent medical data from the medical image data. Pertinent medical data may comprise text data and image data that is related to the medical examination and/or medical research. Further, the pertinent medical data may include a patient identifier, a medical imaging technique identifier, an organ system identifier, an anatomical feature identifier, a date and time of when the medical image data was captured, or any combination thereof. Further, the pertinent medical image data may include quantitative or qualitative annotations associated with the medical examination. For example, an ultrasound (i.e. medical image data) of a particular vascular system may include a measurement of peak systolic velocity (PSV) and end diastolic velocity (EDV), along with an annotation of the whether the measurement is nominal or outside a nominal range.

In some examples, the RIDS system may employ object recognition algorithms, such as appearance-based and feature-based methods—to help identify pertinent medical characteristics from the text and images of the medical image data.

At 706, the RIDA system may generate a set of metadata for the medical image data, based at least in part on an analysis of the pertinent medical data. The set of metadata may include a patient identifier, organ system identifier, medical specialty identifier, medical imaging technique identifier, examination identifier, and patient demographic identifiers, or any combination thereof. In doing so, the RIDA system may associate the set of metadata with each corresponding instance of medical image data.

At 708, the RIDA system may selectively remove patient identifiers presented in the medical image data. The purpose of removing patient identifiers is to safeguard patient privacy and ensure that any distribution of the medical image data, for research or other related purposes, does not inadvertently reveal a patient's identity.

At 710, the RIDA system may store the medical image data, along with the corresponding set of metadata, within a data repository accessible by the RIDA system. The data repository may comprise of a patient data repository and an anonymized data repository. Medical image data that includes patient-identifiable data may be stored in the patient data repository. Further, medical image data that has been anonymized (i.e. patient identifiable data has been removed), may be included in the anonymized data repository. In some examples, a medical image data record that is stored within the anonymized data repository may be linked to a corresponding medical image data record in the patient data repository for the purpose of verifying and tracing an authenticity of the medical image data. In this example, the RIDA system may restrict access to the link within the anonymized data repository, via authentication credentials, authentication tokens, and/or so forth.

Further, while patient identifiers may be visually removed from the medical image data, the set of metadata associated with the medical image data may retain the patient identifier for the purpose of verifying and tracing an authenticity of the medical image data. In some examples, the RIDA system may restrict access to patient identifiers within the set of metadata, via authentication credentials, authentication tokens, and/or so forth.

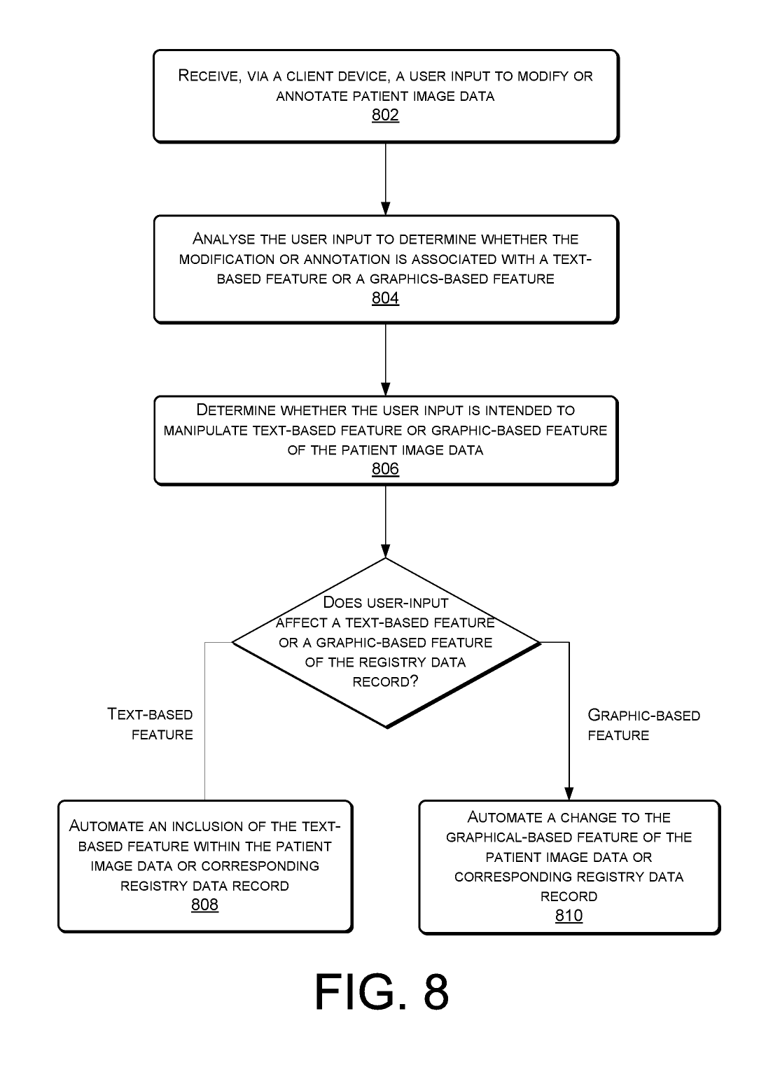

FIG. 8 illustrates a RIDA system process for synthesizing a user input to modify patient image data and/or a corresponding registry data record. The patient image data may be part of a medical examination and/or medical research, and include medical result data from an x-ray scan, magnetic resonance imaging (MRI) scan, computed tomography (CT) scan, ultrasound scan, or a Positron Emission Tomography (PET) scan.

At 802, the RIDA system may receive, via a client device, a user input to modify or annotate patient image data. In some examples, the user input may correspond to an audio-based user input. In other examples, the user input may correspond to a text-based user input. In some examples, a RIDA system may receive the audio-based user input via a RIDA application native on the client device.

At 804, the RIDA system may analyze the user input using natural language processing (NLP) and natural language understanding (NLU) algorithms, or a data mining algorithm, to determine whether the modification or annotation is associated with a text-based feature or a graphics-based feature of the patient image data. For example, the modification or annotation may comprise of an interpretation of medial results (i.e. text information) that is to be overlaid onto patient image data.

In another example, the audio-based user input may be associated with a command to reflect a medical condition on a schematic diagram of an organ system. In the latter example, consider a patient image data that includes a schematic illustration of a patient's cardiovascular system. In this example, the audio-based user input may include a command to reflect a 50% partial constriction, or occlusion, of a carotid artery at a specific location on the schematic illustration.

At 806, the RIDA system may determine whether the user input (i.e. audio-based user input or text-based user input) is intended to manipulate a text-based feature or a graphics-based feature of the patient image data. Text-based features may include annotations associated with a diagnostic interpretation of medical image data and/or medical results. Graphics-based features may include schematic illustrations of an organ system, such as a patient's cardiovascular system, and/or medical results.

At 808, the RIDA system may determine that the user input (i.e. audio-based user input or text-based user input) is intended to manipulate text-based features of the patient image data or corresponding registry data record. In doing so, the RIDA system may automate an inclusion of the text-based feature at a specific text-field location of the patient image data or corresponding registry data record.

At 810, the RIDA system may determine that the user input (i.e. audio-based user input or text-based user input) is intended to manipulate graphics-based features of the patient image data or corresponding registry data record. In doing so, the RIDA system may automate a change to the graphics-based feature.

### CONCLUSION

Although the subject matter has been described in language specific to features and methodological acts, it is to be understood that the subject matter defined in the appended claims is not necessarily limited to the specific features or acts described herein. Rather, the specific features and acts are disclosed as exemplary forms of implementing the claims.

What is claimed:

1. A system comprising:
one or more processors;
memory coupled to the one or more processors, the memory including one or more modules that are executable by the one or more processors to:
receive, from a client device, a request to infer a medical diagnosis associated with patient image data, the request further including the patient image data;
extract a set of pertinent medical data from the patient image data, the set of pertinent medical data includes at least a medical test identifier and medical result data;
retrieve, from a data repository, a set of stored medical image data, based at least in part on the set of pertinent medical data;
analyze a set of image features of the patient image data to identify data patterns with the set of stored medical image data; and
infer the medical diagnosis, based at least in part on analysis of the set of image features of the patient image data.

2. The system of claim 1, wherein, the patient image data further includes text-based result data, and wherein the one or more modules are further executable by the one or more processors to:
analyze the text-based result data to identify data patterns with the set of stored medical image data, and
wherein, to infer the medical diagnosis associated with the patient image data is further based at least in part on analysis of the text-based result data.

3. The system of claim 1, wherein the one or more modules are further executable by the processors to:
retrieve registry data records associated with the set of stored medical image data, registry data records including medical interpretations of corresponding medical image data, and
wherein, to infer a medical condition is further based at least in part on the registry data records.

4. The system of claim 1, wherein the one or more modules are further executable by the one or more processors to:
receive, an additional request to validate a medical hypothesis associated with the patient image data, the additional request including a first set of criteria and a second set of criteria, the first set of criteria identifying profile data for a control group of patients associated with the medical hypothesis, and the second set of criteria including one or more test algorithms to test the medical hypothesis;
retrieve an additional set of stored medical image data based at least in part on the first set of criteria;
analyze the additional set of stored medical image data, based at least in part on the second set of criteria; and
infer a validity of the medical hypothesis, based at least in part on analysis of the additional set of stored medical image data.

5. The system of claim 4, wherein the set of stored medical image data is a first set of stored medical image data, and wherein the one or more modules are further executable by the one or more processors to:
retrieving, from the data repository, a second set of stored medical image data associated with a predetermined patient population captured over a predetermined time interval, the first set of stored medical image data being a subset of the second set of stored medical image data; and
generate a statistical data model based at least in part on analyses of the second set of stored medical image data, and
wherein, to infer the validity of the medical hypothesis is based at least in part on the statistical data model.

6. The system of claim 4, wherein the one or more modules are further executable by the one or more processors to:
generate validity scores for individual analyses of the one or more test algorithms; and
identify a subset of the individual analyses that have individual validity scores greater than or equal to a predetermined validity threshold, and
wherein to infer the validity of the medical hypothesis is further based at least in part on the subset of the individual analyses.

7. The system of claim 1, wherein the one or more modules are further executable by the one or more processors to:
receive, via the client device, a user input to modify or annotate the patient image data, the user input corresponding to an audio-based user input or a text-based user input;
determine whether the user input is associated with modification or annotation of a text-based feature or a graphics-based feature of the patient image data; and
generate an updated patient image data by automatically modifying or annotating the patient image data, based at least in part on the user input, and
wherein, to analyze the set of image features of the patient image data includes analysis of the updated patient image data.

8. The system of claim 1, wherein the one or more modules are further executable by the one or more processors to:
capture, from one or more vendors, additional medical image data for inclusion in the data repository;
analyze, via one or more machine learning algorithms, the additional medical image data to identify patient identifiers;
selectively remove the patient identifiers from individual ones of the additional medical image data; and
store, within the data repository, the additional medical image data, and
wherein, to retrieve the set of stored medical image data includes the additional medical image data.

9. The system of claim 8, wherein the one or more modules are further executable by the one or more processors to:
extract an additional set of pertinent medical data for the individual ones of the additional medical image data;
generate a set of metadata for the individual ones of the additional medical image data, based at least in part on the additional set of pertinent medical data; and
associate the set of metadata with the individual ones of the additional medical image data.

10. The system of claim 1, wherein the set of image features of the patient image data include a graphical depiction of anatomical features of an organ system or graphics-based result data.

11. The system of claim 1, wherein the set of stored medical image data is associated with one or an x-ray test, a magnetic resonance imaging (MRI) scan, a computed tomography (CT) scan, an ultrasound, or a Positron Emission Tomography (PET) scan.

12. A computer-implemented method, comprising:
under control of one or more processors:
receiving, from a client device, a request to infer a validity of a medical hypothesis associated with patient image data, the request including a set of control group criteria identifying profile data for a control group of patients associated with the medical hypothesis;
retrieving, from a data repository, a set of stored medical image data, based at least in part on the set of control group criteria;
analyzing a set of image features associated with individual ones of the set of stored medical image data to identify a correlation with the medical hypothesis; and
inferring the validity of the medical hypothesis based at least in part on analysis of the set of stored medical image data.

13. The computer-implemented method of claim 12, further comprising:
extracting a set of pertinent medical data from the patient image data, the set of pertinent medical data including at least a medical test identifier and medical results data; and
generating at least one hypothesis rule based at least in part on the medical hypothesis, the at least one hypothesis rule correlating the validity of the medical hypothesis with a subset of the set of pertinent medical data, and
wherein, analyzing the set of stored medical image data is based at least in part on the at least one hypothesis rule.

14. The computer-implemented method of claim 12, wherein the set of stored medical image data is a first set of stored medical image data, and further comprising:
retrieving, from the data repository, a second set of stored medical image data associated with a predetermined patient population captured over a predetermined time interval, the first set of stored medical image data being a subset of the second set of stored medical image data; and
generating a statistical data model based at least in part on analyses of the second set of stored medical image data, and
wherein, inferring the validity of the medical hypothesis is based at least in part on statistical data model.

15. The computer-implemented method of claim 14, further comprising:
determining that the medical hypothesis is not valid;
modifying, via one or more machine-learning algorithms, the set of control group criteria to create a modified set of control group criteria, based at least in part on the statistical data model;
retrieving, from the data repository, an additional set of stored medical image data, based at least in part on the modified set of control group criteria; and
inferring the validity of the medical hypothesis based at least in part an additional analysis of the additional set of stored medical image data.

16. The computer-implemented method of claim 12, wherein, the request further includes an additional set of criteria that includes one or more test algorithms to test the medical hypothesis, and further comprising:
generating validity scores for individual analyses of the one or more test algorithms; and
identifying a subset of the individual analyses that have individual validity scores greater than or equal to a predetermined validity threshold, and
wherein, to infer the validity of the medical hypothesis is further based at least in part on the subset of the individual analyses.

17. One or more non-transitory computer-readable media storing computer-executable instructions that, when executed on one or more processors, cause the one or more processors to perform acts comprising:
receiving, from a client device, a request to infer a medical hypothesis associated with patient image data, the request further including the patient image data;
extracting pertinent medical image data from the patient image data;
generating at least one hypothesis rule based at least in part on the medical hypothesis, the at least one hypothesis rule correlating a validity of the medical hypothesis with a subset of the pertinent medical image data;
analyzing a set of image features associated with individual ones of a set of stored medical image data, based at least in part on the at least one hypothesis rule; and
inferring the validity of the medical hypothesis based at least in part on analysis of the set of image features.

18. The one or more non-transitory computer-readable media of claim 17, wherein the request further includes a first set of criteria and a second set of criteria, the first set of criteria identifying profile data for a control group of patients associated with the medical hypothesis, and the second set of criteria including one or more test algorithms to test the medical hypothesis, and further storing instructions that, when executed cause the one or more processors to perform acts comprising:
retrieving, from a data repository, the set of stored medical image data based at least in part on the first set of criteria, and
wherein, analyzing the set of image features further is further based at least in part on the second set of criteria.

19. The one or more non-transitory computer-readable media of claim 18, further storing instructions that, when executed cause the one or more processors to perform acts comprising:
determining that the medical hypothesis is not valid, based at least in part on analysis of the set of image features;
modifying, via one or more machine-learning algorithms, the first set of criteria to create a modified first set of criteria;
retrieving, from the data repository, an additional set of stored medical image data, based at least in part on the modified first set of criteria;
analyzing the additional set of stored medical image data based at least in part on the second set of criteria; and
inferring the validity of the medical hypothesis based at least in part on an additional analysis of the additional set of stored medical image data.

20. The one or more non-transitory computer-readable media of claim 17, further storing instructions that, when executed cause the one or more processors to perform acts comprising:
capturing, from one or more vendors, vendor medical image data for inclusion in a data repository;
selectively removing patient identifiers from individual ones of the vendor medical image data;
storing, within the data repository, the vendor medical image data as the set of stored medical image data; and
retrieving, from the data repository, the set of stored medical image data.
